(19) United States
(12) Patent Application Publication
(10) Pub. No.: US 2019/0076357 A1
(43) Pub. Date: Mar. 14, 2019

(54) LIPOSOMAL PACLITAXEL PALMITATE FORMULATION AND PREPARATION METHOD THEREOF

(71) Applicant: SHANGHAI WEI ER BIOPHARMACEUTICAL TECHNOLOGY CO., LTD., Shanghai (CN)

(72) Inventors: Bin SHAN, Shanghai (CN); Qinqin ZHOU, Shanghai (CN); Nong YU, Shanghai (CN)

(21) Appl. No.: 16/185,020

(22) Filed: Nov. 9, 2018

Related U.S. Application Data
(63) Continuation-in-part of application No. PCT/CN2017/082356, filed on Apr. 28, 2017.

(30) Foreign Application Priority Data
May 9, 2016 (CN) 201610301096.4

(51) Int. Cl. A61K 9/127; A61K 9/19; A61K 9/08; A61K 31/337; A61K 9/00; A61K 47/24; A61K 47/28; A61K 47/26; A61K 47/02

(52) U.S. Cl. CPC A61K 9/127; A61K 9/19; A61K 9/08; A61K 9/1277; A61K 47/02; A61K 9/0019; A61K 47/24; A61K 47/28; A61K 47/26; A61K 31/337

(57) ABSTRACT

A liposomal paclitaxel palmitate formulation, including: 0.1-1% (w/v) of a paclitaxel palmitate; 1-10% (w/v) of a lecithin; 0.05-1.0% (w/v) of distearoyl phosphoethanolamine-polyethylene glycol 2000 (DSPE-PEG2000); and water. A method for preparing the formulation includes: mixing the paclitaxel palmitate, lecithin, cholesterol, DSPE-PEG2000, and an organic solvent, and heating the resulting mixture at 25-75° C. to yield an organic phase; heating an aqueous phase to 25-75° C., and stirring and adding the organic phase to the aqueous phase, to yield a crude liposome; emulsifying the crude liposome, to yield a liposome solution; adding a cryoprotector to the liposome solution, adding water to the liposome solution to reach a preset calibration, adding a pH modifier, filtering the liposome solution, and packaging.

### CROSS-REFERENCE TO RELATED APPLICATIONS

This application is a continuation-in-part of International Patent Application No. PCT/CN2017/082356 with an international filing date of Apr. 28, 2017, designating the United States, now pending, and further claims foreign priority benefits to Chinese Patent Application No. 201610301096.4 filed May 9, 2016. The contents of all of the aforementioned applications, including any intervening amendments thereto, are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, Cambridge, Mass. 02142.

### BACKGROUND

This disclosure relates to a liposomal paclitaxel palmitate formulation and a preparation method thereof.

Paclitaxel (PTX) is a clinical drug used to treat a number of cancers such as ovarian cancer, breast cancer, lung cancer, Kaposi sarcoma, cervical cancer, and pancreatic cancer.

Typically, the use of paclitaxel brings the side effects including but not limited to hair loss, bone marrow suppression, allergic reactions, muscle pains, and diarrhea.

### SUMMARY

The disclosure provides a liposomal paclitaxel palmitate formulation, and a method for preparing the same.

The inventors conducted a series of comparison experiments with regard to the antitumor effect of C₆₋₂₆ paclitaxel fatty acid esters, it was found that the paclitaxel palmitate exhibited strong antitumor effect. At a dosage of 15 mg/kg (based on paclitaxel), the tumor inhibition rate was more than 70% versus 50% or even lower than 50% for the other C₆-C₂₆ paclitaxel fatty acid esters.

During the dose-form study, the inventors discovered that the anti-tumor effects of nano-formulations such as polymer micelles, fat emulsions, and nanoparticles were not effective as those of liposomes, because nano-formulations have a series of defects such as low drug loading, drug precipitation, inability to remove bacteria, and poor efficacy.

Common preparation methods of the liposomal paclitaxel palmitate formulation include thin film evaporation method, reverse evaporation method, and ethanol injection method. The thin film evaporation method has poor controllability and complicated steps. As for paclitaxel palmitate, neither the reverse evaporation method nor the ethanol injection method can achieve effective liposomes. The obtained liposomes have a series of problems including low drug loading, turbidity, precipitation, and inability to remove bacteria. It was found that the use of propylene glycol instead of ethanol injection can achieve a practical effect. Therefore, use of the solvent of propylene glycol or propylene glycol is the core technical feature of the method for preparing the liposomal paclitaxel palmitate formulation. This is quite different from the preparation of conventional liposomes. In addition, propylene glycol is a small molecule of lower alcohol, which is highly safe and widely used as a solvent. In the disclosure, propylene glycol is used as the solvent. The final liposome may also contain propylene glycol, which may also be separated and removed by ultrafiltration. Hence, the addition of propylene glycol does not affect the relevant properties of the preparation.

The disclosure provides a liposomal paclitaxel palmitate formulation comprising lecithin or a mixture of lecithin and cholesterol. As for the liposome, DSPE-PEG2000 must be added to the formulation at an appropriate amount, or stable liposomes cannot be prepared.

Provided is a liposomal paclitaxel palmitate formulation that comprises 0.1-1% (w/v) of a paclitaxel palmitate; 1-10% (w/v) of a lecithin; 0.05-1.0% (w/v) of distearoyl phosphoethanolamine-polyethylene glycol 2000 (DSPE-PEG2000); and water.

The amount of the DSPE-PEG2000 is particularly between 0.1% (w/v) and 0.5% (w/v).

The liposomal paclitaxel palmitate formulation is a prodrug of paclitaxel, the structure of which is shown in formula I below:

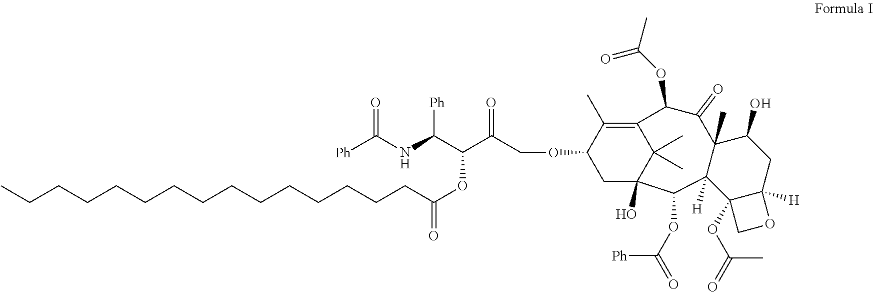 [SMILES: CCCCCCCCCCCCCCCC(=O)O[C@@H](C(=O)CO[C@H]1C[C@@]2(O)[C@@H](OC(=O)c3ccccc3)[C@@H]3[C@]4(OC(C)=O)CO[C@@H]4C[C@H](O)[C@@]3(C)C(=O)[C@H](OC(C)=O)C(=C1C)C2(C)C)[C@@H](NC(=O)c1ccccc1)c1ccccc1]

The paclitaxel palmitate is obtained by esterifying palmitic acid with the 2′ hydroxyl group of paclitaxel.

The paclitaxel palmitate can be obtained by the following synthetic steps:

10.00 g paclitaxel, 3.60 g palmitic acid, 2.18 g 1-(3-dimethylaminopropyl)-3-ethylcarbodiimide (EDC), and 1.72 g 4-dimethylaminopyridine (DMAP) are placed in a reaction vessel and dissolved with 50 ml anhydrous dichloromethane. Under a nitrogen blanket, the reaction is stirred at room temperature for 4-24 hours to obtain a solution, which is washed twice with a 5% aqueous citric acid solution, and then washed once with a saturated sodium chloride solution, and evaporated to remove anhydrous dichloromethane by rotary evaporation. Finally, paclitaxel palmitate is obtained after separation and purification.

The liposomal paclitaxel palmitate formulation can be in the form of an aqueous injection, or a lyophilized powder.

The formulation can comprise paclitaxel palmitate and lecithin; the formulation can comprise paclitaxel palmitate, lecithin and DSPE-PEG2000; the formulation consists of paclitaxel palmitate, high-purity egg yolk lecithin (EPCS) and DSPE-PEG2000; the formulation can comprise paclitaxel palmitate, high-purity EPCS, DSPE-PEG2000 and a cryoprotector.

The formulation can comprise:

| | | |
|---|---|---|
| Paclitaxel palmitate | 0.1-1% | g/mL; |
| Lecithin | 1-10% | g/mL; |
| Cholesterol | 0-1% | g/mL; |
| DSPE-PEG2000 | 0.05-1.0% | g/mL; |
| Cryoprotector | 0-40% | g/mL; |
| pH modifier adapting to | | |
| regulate a pH value of the | | |
| formulation to 3.0-9.0; and | | |
| Water. | | |

When preparing a solution for formulation, the dosage of cryoprotector can be 0.

When preparing a lyophilized powder formulation, the dosage of cryoprotector can be 0.1-40% g/mL.

The formulation can comprise:

| | | |
|---|---|---|
| Paclitaxel palmitate | 0.1-0.7% | g/mL; |
| Lecithin | 1-8% | g/mL; |
| Cholesterol | 0-0.5% | g/mL; |
| DSPE-PEG2000 | 0.1-0.8% | g/mL; |
| Cryoprotector | 5-40% | g/mL; |
| pH modifier adapting to | | |
| regulate a pH value of the | | |
| formulation to 4.0-8.0; and | | |
| Water. | | |

The formulation can comprise:

| | | |
|---|---|---|
| Paclitaxel palmitate | 0.2-0.5% | g/mL; |
| High purity EPCS | 2-6% | g/mL; |
| Cholesterol | 0-0.5% | g/mL; |
| DSPE-PEG2000 | 0.1-0.5% | g/mL; |
| Cryoprotector | 10-35% | g/mL; |
| pH modifier adapting to | | |
| regulate a pH value of the | | |
| formulation to 4.5-7.5; and | | |
| Water. | | |

The lecithin can be selected from the group consisting of egg phosphatidyl choline (EPCS), hydrogenated soya lecithin, 1,2-dimyristoyl-sn-glycero-3-phosphocholine (DMPC), soyabean lecithin, 1,2-dipalmitoyl-sn-glycero-3-phosphocholine (DPPC), 1,2-distearoyl-sn-glycero-3-phosphocholine (DSPC), phosphatidylcholine, egg yolk phosphatidylcholines, phosphatidylethanolamines, phosphatidylserines, sphingomyelin, or a mixture thereof. High purity egg yolk lecithin (EPCS) and hydrogenated soy lecithin (HSPC) are preferred.

The cryoprotector can be selected from the group consisting of maltose, trehalose, sucrose, mannitol, lactose, glucose, sorbitol, xylitol, erythritol, threonine, or a mixture thereof.

The pH modifier is selected from the group consisting of citric acid, hydrochloric acid, sodium hydroxide, phosphoric acid, disodium hydrogen phosphate, sodium dihydrogen phosphate, dipotassium hydrogen phosphate, potassium dihydrogen phosphate, disodium citrate, trisodium citrate, or a mixture thereof. Preferably, one of citric acid, hydrochloric acid, sodium hydroxide or a mixture thereof is used to adjust the pH to 3-9. Preferably, the pH is adjusted to 4-8; more preferably, the pH is adjusted to 4.5-7.5.

A method for preparing a liposomal paclitaxel palmitate formulation comprising 0.1-1% (w/v) of a paclitaxel palmitate, 1-10% (w/v) of a lecithin, and 0.05-10% (w/v) of distearoyl phosphoethanolamine-polyethylene glycol 2000 (DSPE-PEG2000), the method comprises:
1) mixing the paclitaxel palmitate, lecithin, cholesterol, DSPE-PEG2000, and an organic solvent, and heating a resulting mixture at 25-75° C. to yield an organic phase;
2) providing and heating an aqueous phase to 25-75° C., and stirring and adding the organic phase to the aqueous phase, to yield a crude liposome;
3) emulsifying the crude liposome, to yield a liposome solution;
4) adding a cryoprotector to the liposome solution, adding water to the liposome solution to reach a preset calibration, adding a pH modifier, filtering the liposome solution, and packaging.

The organic solvent can be selected from the group consisting of propylene glycol or anhydrous ethanol and tert-butanol, and the amount can be 1-10% g/mL. Propylene glycol can be preferred, and a preferred amount can be 1-5% g/mL.

The organic solvent can be retained in the liposome or removed by ultrafiltration after emulsification of the crude liposome. The liposome particle size distribution of the liposome crude product emulsified by the extrusion emulsification method is uniform. The crude liposome is emulsified by using an extrusion film having a pore size of 2.0 μm, 1.0 μm, 0.8 μm, 0.6 μm, 0.4 μm, 0.2 μm, 0.1 μm or 0.05 μm. One or more extrusion films are used, preferably 0.6 μm, 0.4 μm, 0.2 μm, 0.1 μm, and 0.05 μm.

The paclitaxel palmitate, the lecithin, and the DSPE-PEG2000 can form a liposome having a particle size of 70-130 nm.

The disclosure also provides the use of the above liposomal paclitaxel palmitate formulation for the preparation of an antitumor drug.

When the liposomal paclitaxel palmitate formulation is administered at a dosage of 15 mg/kg (based on paclitaxel), the tumor inhibition rate is more than 70%.

The tumor described above is the tumor treated with paclitaxel and its derivatives, including but not limited to ovarian cancer, breast cancer, lung cancer, colorectal cancer, melanoma, head and neck cancer, lymphoma, and brain tumor.

The advantages of the liposomal paclitaxel palmitate formulation as described in the disclosure are as follows:

(1) The liposomal paclitaxel palmitate formulation does not contain solubilizing agents such as polyoxyethylene castor oil and Tween 80. Hence, the allergic reaction caused by polyoxyethylene castor oil is avoided.

(2) The anti-tumor effect of the liposomal paclitaxel palmitate formulations described in this disclosure is significantly better than that of Taxol.

(3) The liposomal paclitaxel palmitate formulation exhibits sustained-release effect, slowing down the elimination rate of the drug in the body and increasing the concentration of the drug targeted to the tumor site.

(4) The liposomal paclitaxel palmitate formulation can be prepared into the dosage forms of injections and lyophilized powders, which is easy to store.

### BRIEF DESCRIPTION OF THE DRAWINGS

FIG. 1 shows comparison of pharmacodynamics of paclitaxel hexanoate, paclitaxel octanoate, paclitaxel phthalate, paclitaxel laurate, paclitaxel myri state and liposomal paclitaxel palmitate formulation against mouse S180 solid tumor;

FIG. 2 shows comparison of pharmacodynamics of paclitaxel palmitate, paclitaxel stearate, paclitaxel arachidate, paclitaxel behenate, paclitaxel lignin ester and paclitaxel wax ester liposome against mouse S180 solid tumor; and

FIG. 3 shows comparison of pharmacodynamics of various paclitaxel palmitate nanoformulations against mouse S180 solid tumor.

### DETAILED DESCRIPTION

To further illustrate, embodiments detailing a liposomal paclitaxel palmitate formulation are described below. It should be noted that the following embodiments are intended to describe and not to limit the disclosure.

### Example 1

Comparative Evaluation of in Vivo Efficacy Test of Different Series of Paclitaxel Fatty Acid Esters

The paclitaxel described in the Chinese patent CN1202166A is an alpha-bromo fatty acid ester as a paclitaxel fatty acid ester prodrug with a carbon chain length between C₆ and C₁₆. The paclitaxel fatty acid ester and its preparation described in the U.S. patent (U.S. Pat. No. 7,235,583 B1) and International Patent (WO 00/53231) is a fatty acid with a length of C₈-C₂₆. These patents described a paclitaxel fatty acid ester prodrug with a fatty acid carbon chain length between C₆ and C₂₆ but did not make any differentiation study on the length of these fatty acid carbon chains within this range and therefore were inevitably unable to obtain an optimal paclitaxel. The systematic and parallel comparison study on linear fatty acids with a carbon chain length of C₆-C₂₆ was conducted. To ensure the stability of long-term storage, saturated linear fatty acids was selected for esterification with paclitaxel.

In this embodiment, 11 different fatty acid carbon chains of paclitaxel in the length of carbon chain between C₆-C₂₆ were randomly designed and synthesized: paclitaxel hexanoate (C₆), paclitaxel octanoate (C₈), paclitaxel phthalate (C₁₀), paclitaxel laurate (C₁₂), paclitaxel myristate (C₁₄), paclitaxel palmitate (C₁₆), paclitaxel stearate (C₁₈), paclitaxel arachidate (C₂₀), paclitaxel behenate (C₂₂) Paclitaxel lignin ester (C₂₄) and paclitaxel wax ester (C₂₆), and their antitumor effects were compared in parallel.

Preparation of a Series of Different Paclitaxel Fatty Acid Ester Liposomes

Under the same process conditions, liposomes of different paclitaxel fatty acid esters were prepared. To ensure the feasibility and parallelism of the preparation as much as possible, a conservative drug loading amount was set, and the drug loading amount was about 1 mg/mL according to paclitaxel.

An appropriate amount of paclitaxel fatty acid ester (about 100 mg according to paclitaxel), 2 g high-purity egg yolk lecithin (EPCS), 0.3 g DSPE-PEG2000 add 2 g propylene glycol were weighed out, dissolved by heating at 60° C. to obtain the organic phase. 90 g water was heated to 50° C., in which the mixture was dissolved by stirring to obtain an aqueous phase. The organic phase was mixed with the aqueous phase by stirring to obtain a crude liposome. The crude liposome was placed in an extruder, sequentially passed through an extrusion film with a pore sizes of 0.6 μm, 0.4 μm, 0.2 μm, 0.1 μm, and 0.05 μm, diluted with water to 100 mL, and sterilized by filtration through a 0.22 μm filter to obtain a series of paclitaxel fatty acid ester liposomes with different carbon chain lengths.

Parallel Comparison of the Inhibitory Effect of Different Paclitaxel Fatty Acid Esters in S180 Tumor-Bearing Mice

The 11 paclitaxel fatty acid ester liposomes prepared above were used for pharmacodynamic comparison in animals administered the positive control drug Taxol. The test plan and results are as follows:

Establishment of a Mouse S180 Tumor Model and Design of a Dosing Regimen

In vitro cultured S180 tumor cells (Shanghai Institute of Life Sciences, Chinese Academy of Sciences, Shanghai, China) were inoculated into the peritoneal cavity of the mice. The ascites cells thus formed were extracted and diluted with normal saline (NS) to a concentration of 1×10⁶ cells/ml to obtain an ascites cell dilution. 0.2 ml ascites cell dilution was injected subcutaneously into the right forelimb of the Kunming mice to establish a mouse S180 tumor model.

Twenty-four S180 tumor modeled mice were equally randomized to three groups: a NS group, a Taxol positive control group, and a paclitaxel fatty acid liposome test group. Animals in the three groups were administered with NS, Taxol and paclitaxel fatty acid liposome respectively at a dosage of 15 mg/kg each via the tail vein on alternative days, totaling 4 doses. The mice were sacrificed the next day after termination of drug administration. The tumor was removed, weighed and calculated for the tumor inhibition rate. As there were altogether 11 paclitaxel fatty acid ester liposome preparations for test, the experiments were divided into two parts: Comparative Test 1 and Comparative Test 2.

Tumor inhibition rate=(tumor weight in NS group−tumor weight in the drug group)/tumor weight in NS group×100%

The Anti-Tumor Effect of Comparative Test 1

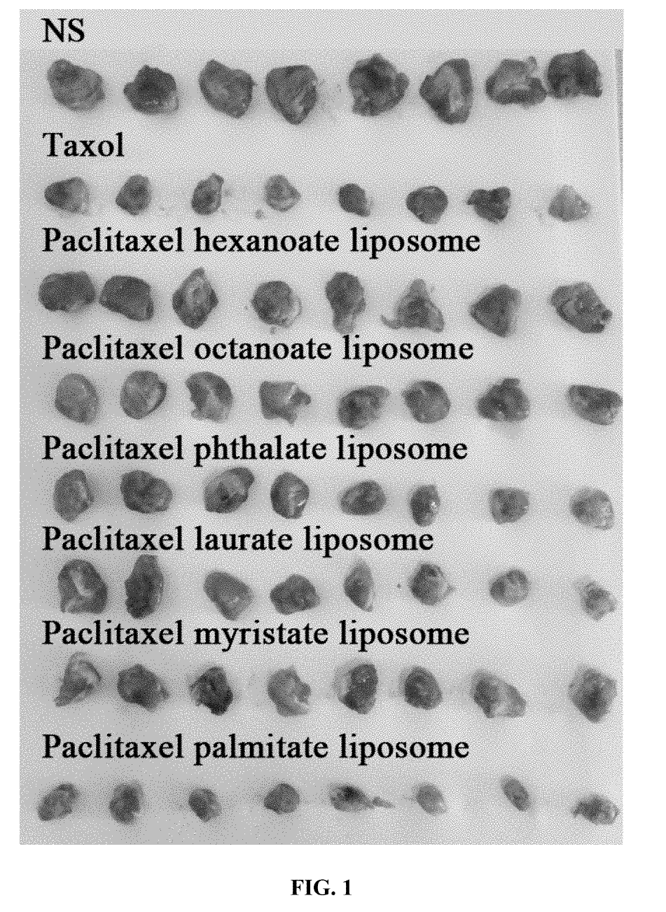

The pharmacodynamic comparison results for mouse S180 solid tumors are shown in Table 1, and the tumor photographs are shown in FIG. 1.

TABLE 1
Comparison of inhibitory effects of different paclitaxel fatty
acid esters on S180 tumor-bearing mice in Test 1

| | Length of | | |
|---|---|---|---|
| | Carbon | Tumor | Tumor inhi- |
| Group | chain | weight (g) | bition rate |
| NS | / | 1.46 ± 0.58 | / |
| Taxol | / | 0.52 ± 0.32 | 64.38% |
| Paclitaxel hexanoate | C6 | 1.03 ± 0.53 | 29.45% |
| liposome | | | |
| Paclitaxel octanoate | C8 | 0.97 ± 0.66 | 33.56% |
| liposome | | | |
| Paclitaxel phthalate | C10 | 0.81 ± 0.47 | 44.52% |
| liposome | | | |
| Paclitaxel laurate liposome | C12 | 0.88 ± 0.35 | 39.73% |
| Paclitaxel myristate | C14 | 0.75 ± 0.29 | 48.63% |
| liposome | | | |
| Liposomal paclitaxel | C16 | 0.32 ± 0.19 | 78.08% |
| palmitate formulation | | | |

The antitumor effect of paclitaxel hexanoate liposomes (C₆), paclitaxel octanoate liposomes (C₈), paclitaxel laurate liposomes (C₁₂), paclitaxel myristate liposomes (C₁₄), and liposomal paclitaxel palmitate formulations (C₁₆) were compared under the same liposome dose form and the same drug loading conditions. Unexpectedly, only the anti-tumor effect of liposomal paclitaxel palmitate formulations was the most prominent, with a tumor inhibition rate of 78.08% versus less than 50% for the other paclitaxel fatty acid esters.

The Anti-Tumor Effect of Comparative Test 2

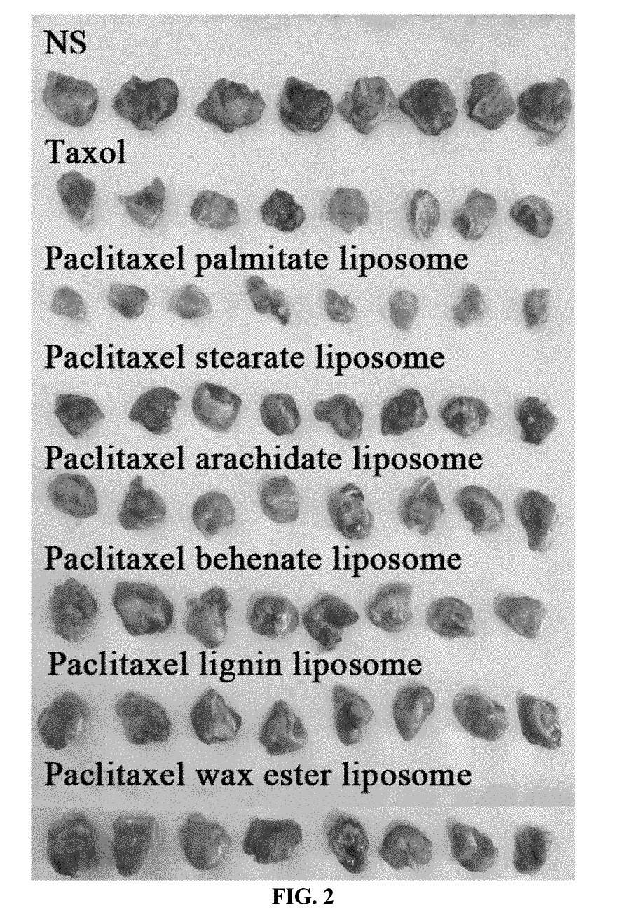

The pharmacodynamic comparison results for mouse S180 solid tumors are shown in Table 2, and the tumor photographs are shown in FIG. 2.

TABLE 2
Comparison of the inhibitory effects of different paclitaxel
fatty acid esters on S180 tumor-bearing mice in Test 2

| | Length of | | |
|---|---|---|---|
| | Carbon | Tumor | Tumor inhi- |
| Group | chain | weight (g) | bition rate |
| NS | / | 1.69 ± 0.65 | / |
| Taxol | / | 0.68 ± 0.44 | 59.76% |
| Liposomal paclitaxel | C₁₆ | 0.43 ± 0.28 | 74.56% |
| palmitate formulation | | | |
| Paclitaxel stearate | C₁₈ | 0.89 ± 0.41 | 47.34% |
| liposome | | | |
| Paclitaxel arachidate | C₂₀ | 1.01 ± 0.39 | 40.24% |
| liposome | | | |
| Paclitaxel behenate | C₂₂ | 1.12 ± 0.46 | 33.73% |
| liposome | | | |
| Paclitaxel lignin | C₂₄ | 0.98 ± 0.55 | 42.01% |
| liposome | | | |
| Paclitaxel wax ester | C₂₆ | 1.21 ± 0.70 | 28.40% |
| liposome | | | |

The antitumor effect of liposomal paclitaxel palmitate formulation (C₁₆), paclitaxel stearate liposome (C₁₈), paclitaxel arachidate liposome (C₂₀), paclitaxel behenate liposome (C₂₂), paclitaxel lignin liposome (C₂₄) and paclitaxel wax ester liposome (C₂₆) were compared under the same liposome dose form and the same drug loading conditions. The results showed that only liposomal paclitaxel palmitate formulations had the best anti-tumor effect, with a tumor inhibition rate of 74.56% versus less than 50% for the other paclitaxel fatty acid esters, further confirming that at the antitumor activity of paclitaxel palmitate was significantly different from that of the other fatty acid esters.

### Example 2

Study on Drug-Forming Properties of Different Formulations and Dose Forms of Paclitaxel Palmitate

Comparative study on drug-forming properties and anti-tumor effects of liposomes, polymer micelles, fat emulsions and nanoparticles were carried out. For the sake of conservatism, the drug loading was fixed at 3 mg/mL for parallel comparison. By referring to the current clinical dosage of paclitaxel (about 400 mg per adult), 3 mg/mL is known as the lower requirement for drug loading. Based on this, the research on related preparations was carried out, and the main research plans and results are summarized as follows:

1. Study on Transition of the Preparation into a Medicine

1.1 Liposomes

300 mg paclitaxel palmitate, 3.5 g high-purity egg yolk lecithin (EPCS), and 0.3 g DSPE-PEG2000 were weighed out, added with 3 g propylene glycol, and dissolved at 65° C. to obtain an organic phase; 85 g water was weighed out, and dissolved by stirring and heated at 65° C. to obtain an aqueous phase. The organic phase was injected into the aqueous phase by stirring, and mixed to obtain a crude liposome, which was then placed in an extruder, and sequentially extruded through an extrusion film with a pore size of 0.4 μm, 0.2 μm, 0.1 μm, and 0.05 μm, and added with water to 100 ml. pH was adjusted to 4.5 with hydrochloric acid. The filter was sterilized by filtration through a 0.22 μm filter to obtain liposomal paclitaxel palmitate formulations.

The liposome was determined to be translucent, with a labeled content of 99.69%, a mean particle size of 102.4 nm, and an entrapment efficiency of 99.45%. The sterilization filtration was quite smooth, and through proper adjustment of the preparatory process, the drug loading can reach 10 mg/mL with good stability without the phenomenon of turbidity, precipitation or increase in particle size. Therefore, the results showed that the liposome was able to wrap the paclitaxel palmitate well, and the liposomal paclitaxel palmitate formulation was good in drug formation.

1.2 Polymer Micelles

Paclitaxel polymer micelle (Genexol®-PM), which was launched in Korea in 2007, is a micelle preparation prepared by wrapping paclitaxel with a high molecular polymer material, polyethylene glycol monomethyl ether-polylactic acid. The experiments with paclitaxel palmitate encapsulation was carried out by setting the weight ratio of paclitaxel palmitate to polyethylene glycol monomethyl ether-polylactic acid at 1:1, 1:5, 1:10, 1:20 and 1:30.

0.3 g paclitaxel palmitate and 0.3 g (or 1.5 g, or 3 g, or 6 g, or 9 g) polyethylene glycol monomethyl ether-polylactic acid were weighed out and dissolved in an appropriate amount of acetonitrile. Acetonitrile was removed by evaporation under reduced pressure, dissolved with 90 ml water, and diluted to 100 mL to obtain paclitaxel palmitate polymer micelles with different polymer materials.

The results showed that the weight ratio of paclitaxel palmitate to polyethylene glycol monomethyl ether-polylactic acid was from 1:1-1:30 at the drug loading of 3 mg/mL, meaning that the amount of polyethylene glycol monomethyl ether-polylactic acid was 0.3-9%. The prepared paclitaxel palmitate polymer micelles were completely turbid, and there was no sign of any wrapping. For paclitaxel, the ratio of 1:1 was completely clarified under the same conditions. These results showed that the physicochemical properties of paclitaxel and paclitaxel palmitate were completely different, and suggested that polyethylene glycol monomethyl ether-polylactic acid was not suitable for encapsulation of paclitaxel palmitate, and therefore paclitaxel palmitate was unlikely to be prepared into polymer micelles.

1.3 Fat Emulsion

After paclitaxel was prepared as paclitaxel palmitate, the solubility in the medium chain oil increased from less than 5 mg/mL to more than 500 mg/mL as compared with paclitaxel, making it possible to prepare high drug-loaded fat emulsions.

0.3 g paclitaxel palmitate and 3 g medium-chain oil were dissolved by stirring and heated at 60° C. to obtain an oil phase; 3 g egg yolk lecithin and 2.5 g glycerin were dispersed in 80 g water, and heated to 60° C. to obtain an aqueous phase; the oil phase was slowly injected into the aqueous phase under 5-min shearing conditions, and added with water to 100 mL to obtain a colostrum, which was then placed in a high-pressure homogenizer, homogenized 5 times at 20,000 psi, and sterilize by filtration through a 0.22 μm filter to obtain a paclitaxel palmitate fat emulsion.

The emulsion was found to have a labeled content of 100.2%, with a mean particle diameter of 109.1 nm and an encapsulation efficiency of 99.7%. As the solubility of paclitaxel palmitate in the medium chain oil was large enough, drug loading can be easily adjusted to 10 mg/mL or more with good stability with no delamination, precipitation or increase in particle size, indicating that drug affinity of the fat emulsion thus obtained is good.

1.4 Nanoparticles

Walter el al succeeded in preparing a nanoparticle coated with DSPE-PEG2000 (Walter R. Perkins, Imran Ahmad et al, Novel therapeutic nano-particles (liposomes): trapping poorly water soluble compounds, International Journal of Pharmaceutics 2000, 200: 27-39.). Based on their experience, a nanoparticle of paclitaxel palmitate was prepared. Briefly, 0.3 g paclitaxel palmitate and 0.3 g DSPE-PEG2000 were dissolved in 3 g absolute ethanol to obtain an organic phase; the organic phase was then mixed in 90 ml water of the aqueous phase at 35° C. under stirring and diluted to 100 mL with water; 0.22 μm filter membrane was used for sterilization filtration to obtain a paclitaxel palmitate nanoparticle solution.

The obtained nanoparticle solution was found to be semi-transparent, with a marked percentage content (%) of 99.39 and a particle diameter of 99.4 nm. In addition, the result of the experiment with adjustment of the amount of DSPE-PEG2000 and ethanol showed that the drug loading was higher than 5 mg/mL, indicating that the paclitaxel palmitate nanoparticles have good drug affinity.

In summary, paclitaxel palmitate can be prepared into liposomes, fat emulsions and nanoparticles, all of which can achieve higher drug loading.

2. Anti-Tumor Test Study of Different Preparation Types

The above-prepared liposomal paclitaxel palmitate formulations, fat emulsions and nanoparticles with almost the same drug loading amount and particle diameter were administered to animals for antitumor tests.

S180 tumor cells (Shanghai Institute of Life Sciences, Chinese Academy of Sciences) were cultured in vitro, and then inoculated into the peritoneal cavity of mice to form ascites. S180 tumor ascites cells thus formed were extracted and diluted with NS to a concentration of 1×10⁶ cells/ml to obtain an ascites cell dilution. 0.2 ml ascites cell dilution was injected subcutaneously into the right forelimb of the Kunming mice to obtain a mouse S180 tumor model.

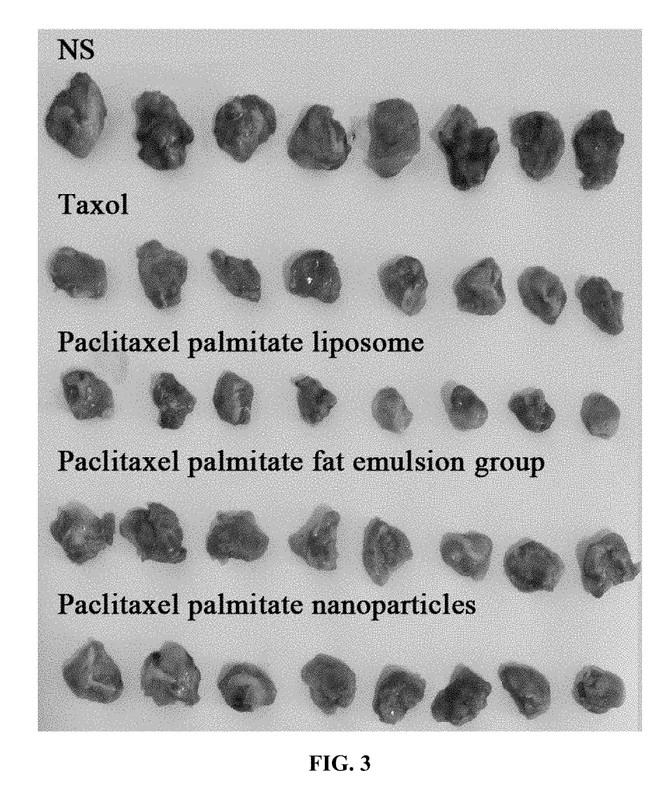

Forty mice were equally randomized to five groups: NS group, Taxol positive control group, liposomal paclitaxel palmitate formulation group, paclitaxel palmitate fat emulsion group, and paclitaxel palmitate nanoparticle group, and administered with respective drugs at a dosage of 15 mg/kg each via the tail vein on alternative days, totaling four doses. The mice were sacrificed the next day after drug administration. Then, the tumor was removed and weighed to calculate the tumor inhibition rate. The results of drug efficacy are shown in Table 3, and the tumor photographs are shown in FIG. 3.

Tumor inhibition rate=(tumor weight in NS group−tumor weight in drug group)/tumor weight in NS group×100%

TABLE 3
Anti-tumor test results of different preparation types of
paclitaxel palmitate

| | Tumor | Tumor inhi- |
|---|---|---|
| Group | weight (g) | bition rate |
| NS | 1.98 ± 0.71 | / |
| Taxol | 0.93 ± 0.41 | 53.03% |
| Liposomal paclitaxel palmitate formulation | 0.59 ± 0.33 | 70.20% |
| Paclitaxel palmitate fat emulsion group | 1.17 ± 0.45 | 40.91% |
| Paclitaxel palmitate nanoparticles | 1.08 ± 0.39 | 45.45% |

Result Analysis:

Surprisingly, the anti-tumor effect of liposomal paclitaxel palmitate formulations was particularly prominent, far better than that of fat emulsion and nanoparticle groups. The tumor inhibition rate of liposomal paclitaxel palmitate formulations was high as 78.2%, which was even better than that of Taxol. To confirm this finding, several experiments were conducted and the results were all the same, indicating that liposome is the best dose-loading form for preparing paclitaxel palmitate into a drug with a good therapeutic effect.

### Example 3

Importance of the Propylene Glycol-Injection Method for Developing Liposomal Paclitaxel Palmitate Formulations

There are many different methods for preparing liposomes, among which the thin-film hydration method, reverse evaporation method and ethanol-injection method are most commonly used. Knowing that the thin-film hydration method has poor controllability and cumbersome preparing steps for large-scale production, the reverse evaporation method and ethanol-injection method were employed to prepare liposomes. However, neither methods can successfully prepare liposomal paclitaxel palmitate formulations compared with the propylene glycol-injection method described in Example 2. The results of liposomal paclitaxel palmitate formulations by the reverse evaporation and ethanol-injection methods are as follows:

1. Reverse Evaporation Method

300 mg paclitaxel palmitate, 3.5 g high-purity egg yolk lecithin (EPCS), 0.3 g DSPE-PEG2000 were dissolved in 10 ml chloroform. The mixture was added with 2 ml water and emulsified by sonication to obtain Calpis. The capias was placed in an eggplant-shaped flask at 37° C. to remove the chloroform by rotary evaporation to form a gel-like liquid. Then, 90 ml water was added to a sufficient amount of gel-like liquid to obtain a crude liposome. The crude liposome was further emulsified in a high-pressure homogenizer and diluted to 100 mL with water to obtain the liposomal paclitaxel palmitate formulation.

The liposome prepared by the reverse evaporation method was not well emulsified and looked turbid, and therefore sterilization filtration was not continued. The mean particle size of the liposome was 268.3 nm with a wide particle size distribution. Further adjustment of the preparatory process and parameters failed to improve the shape and particles size, indicating that the reverse evaporation method was not feasible for large-scale production as compared with the propylene glycol-injection method described in Example 2.

2. Ethanol-Injection Method

300 mg paclitaxel palmitate, 3.5 g high-purity egg yolk lecithin (EPCS) and 0.3 g DSPE-PEG2000 were dissolved in 3 g absolute ethanol by heating to obtain an oil phase; 85 g water was heated to 65° C. to obtain a water phase; the oil phase was mixed with water phase by stirring to obtain the crude liposome; the liposomal paclitaxel palmitate formulation was not obtained after the extrusion process with greater resistance compared with the propylene glycol-injection method. So the crude liposome was further homogenization emulsified with the high-pressure homogenizer, and then filtered by sterilization to obtain a liposomal paclitaxel palmitate formulation.

The test results showed that the liposome prepared by the ethanol-injection method cannot be sterilized by filtration. The particle size of the liposome was 163.1 nm with a wide particle size distribution. The transparency of the preparation was even worse than that of the liposome prepared by the propylene glycol-injection method. Further adjustment of the prescription and parameters of the preparatory technique failed to improve the shape, particles size and sterilization by filtration, indicating that the ethanol-injection method was not suitable for large-scale production as compared with the liposome prepared with propylene glycol-injection method described in Example 2. However, when the ethanol was replaced with ethanol combination with propylene glycol or tert-butanol combination with propylene glycol, liposome was prepared well, indicating that propylene glycol was the key solution for preparing the liposomal paclitaxel palmitate formulation.

In summary, the liposomal paclitaxel palmitate formulation prepared by the conventional reverse evaporation and ethanol-injection methods cannot meet the standard of liposome, probably because of the unique physicochemical properties of paclitaxel palmitate. The obtained liposome was characterized by low drug loading, turbidity, precipitation, and inability to be sterilized by filtration. However, when liposome was prepared by the propylene glycol-injection method, an unexpected effect was obtained, which directly increased the drug affinity of liposome. Therefore, the propylene glycol or binary solution containing propylene glycol was selected as the solvent for preparing the liposomal paclitaxel palmitate formulation. The results showed that the amount of the solvent was 1-10% g/mL, preferably 1-5% g/mL. In addition, propylene glycol is a small molecule lower alcohol widely used as a solvent with high safety. The use of propylene glycol as the solvent is an important part in preparing the liposome described herein. Our experiment showed that the propylene glycol in the liposome can either be retained or removed by ultra-filtration without affecting the relevant properties of the preparation.

### Example 4

Importance of DSPE-PEG2000 in Developing Liposomal Paclitaxel Palmitate Formulation

Usually liposomes are composed of lecithin or lecithin and cholesterol. It is difficult to turn out a stable liposomal paclitaxel palmitate formulation preparation without adding a proper amount of DSPE-PEG2000, no matter how the preparatory process is adjusted, or even when the drug loading is set at 2 mg/mL. The typical verification schemes are shown in Table 4 and the results are shown in Table 5.

1. Prescription Verification

TABLE 4
Prescription design for the test validation protocol

| | Prescription | Prescription | Prescription | Prescription | Prescription | Prescription | Prescription |
|---|---|---|---|---|---|---|---|
| Components | 1 | 2 | 3 | 4 | 5 | 6 | 7 |
| Paclitaxel | 0.2 g | 0.2 g | 0.2 g | 0.2 g | 0.2 g | 0.2 g | 0.2 g |
| palmitate | | | | | | | |
| Egg yolk | 2 g | 4 g | 6 g | 2 g | 2 g | 2 g | 2 g |
| lecithin | | | | | | | |
| (EPCS) | | | | | | | |
| DSPE- | / | / | / | 0.01 g | 0.05 g | 0.1 g | 0.3 g |
| PEG2000 | | | | | | | |
| Propylene | 5 g | 5 g | 5 g | 5 g | 5 g | 5 g | 5 g |
| glycol | | | | | | | |
| Water | to 100 mL | to 100 mL | to 100 mL | to 100 mL | to 100 mL | to 100 mL | to 100 mL |

2. The Preparatory Process

Oil phase was prepared with a prescribed amount of paclitaxel palmitate, egg yolk lecithin (EPCS) or DSPE-PEG2000 and 5 g propylene glycol. The mixture was dissolved upon heating at 65° C. 85 g water was heated to 65° C. to obtain a water phase. The oil phase was mixed with water phase by stirring to obtain a crude liposome. The crude product was further emulsified in a high-pressure homogenizer and diluted to 100 mL with water. The pH value was adjusted to 4.5 with hydrochloric acid. Finally, the liposomal paclitaxel palmitate formulation was sterilized through a 0.22 μm nylon syringe filter.

3. Results

Filter sterilization rather than hot press sterilization was applied to sterilize the liposomal paclitaxel palmitate formulation. The appearance and the mean particle size were analyzed after filtration of the liposome through the 0.22 μm nylon syringe filter, and the filter fluidity was detected during the process of sterilization. The results are shown in Table 5.

TABLE 5
Results of DSPE-PEG2000 on drug-induced properties

| | | Mean particle | |
|---|---|---|---|
| Group | Appearance | size (nm) | filter fluidity |
| Prescription 1 | Turbid emulsion, | 195.7 | Difficult to filter |
| | opaque | | |
| Prescription 2 | Turbid emulsion, | 174.2 | difficult to filter |
| | opaque | | |
| Prescription 3 | Turbid emulsion, | 153.1 | difficult to filter |
| | opaque | | |
| Prescription 4 | translucent | 130.3 | difficult to filter |
| Prescription 5 | Translucent, | 115.4 | easer to filter |
| | homogeneous | | |
| Prescription 6 | Translucent, | 100.2 | ease to filter |
| | homogeneous | | |
| Prescription 7 | Translucent, | 80.7 | ease to filter |
| | homogeneous | | |

4. Result Analysis

When DSPE-PEG2000 was absent, the prepared liposome solution looked turbid; and the particle size was relatively large, it cannot be filtered (such as prescription 1-3). At the same time, translucent and homogeneous liposome cannot be prepared with other types of lipid materials. The prepared liposome can be turbid or precipitate in a short period of time. A translucent and filter sterilizable liposome can be obtained when a small amount of DSPE-PEG2000 was added (more than 0.05%).

It can be concluded that the addition of DSPE-PEG2000 is crucial for the liposome and will directly affect the drug-forming properties of the liposome. This phenomenon was rarely encountered in the preparation of general drug-loaded liposomes, probably due to the unique physicochemical properties of paclitaxel palmitate. Large number of experiments demonstrated that the amount of DSPE-PEG2000 in different prescriptions was 0.05-1.0%, preferably 0.1-0.5%.

### Example 5

Preparation of Liposomal Paclitaxel Palmitate Formulations

Oil phase was prepared with 0.26 g paclitaxel palmitate, 2.9 g high-purity egg yolk lecithin (EPCS), 0.32 g DSPE-PEG2000 and 3 g propylene glycol. The mixture was dissolved upon heating at 65° C. by addition of 68 g water to obtain water phase. The oil phase was mixed with water phase by stirring to obtain a crude liposome. The crude liposome was placed in an extruder, and sequentially passed through a nylon syringe filter of 0.4 μm, 0.2 μm, 0.1 μm and 0.05 μm to obtain liposome solution. 10 g maltose and 15 g trehalose were dissolved in the liposome solution by stirring and diluted to 100 mL with water. The pH value was adjusted to 4.69 with hydrochloric acid. The liposome was filtrated and sterilized through a 0.22 μm nylon syringe filter. Then the filtrate was separately packaged and cap-sealed to obtain a liposomal paclitaxel palmitate formulation with a mean particle size of 100.9 nm.

### Example 6

Preparation of Liposomal Paclitaxel Palmitate Formulations

Oil phase was prepared with 0.26 g paclitaxel palmitate, 3.5 g high-purity egg yolk lecithin (EPCS), 0.35 g DSPE-PEG2000 and 3 g propylene glycol. The mixture was dissolved upon heating at 75° C. 60 g water was heated to 75° C. to obtain a water phase. The oil phase was mixed with water phase by stirring to obtain a crude liposome. The crude liposome was placed in an extruder, and sequentially passed through a nylon syringe filter of 0.4 μm, 0.2 μm, 0.1 μm and 0.05 μm to obtain liposome solution. 15 g maltose, 5 g trehalose and 15 g mannitol were dissolved in the liposome solution by stirring and diluted to 100 mL with water. The pH value was adjusted to 4.70 with citric acid. The liposome was filtrated and sterilized through a 0.22 μm nylon syringe filter. Then, the filtrate was separately packaged and cap-sealed to obtain a liposomal paclitaxel palmitate formulation with a mean particle size of 114.4 nm.

### Example 7

Preparation of Liposomal Paclitaxel Palmitate Formulations

Oil phase was prepared with 0.26 g paclitaxel palmitate, 2.9 g high-purity egg yolk lecithin (EPCS), 0.32 g DSPE-PEG2000 and 3 g propylene glycol. The mixture was dissolved upon heating at 70° C. 65 g water was heated to 75° C. to obtain a water phase. The oil phase was mixed with water phase by stirring to obtain a crude liposome. The crude liposome was placed in an extruder, and sequentially passed through a nylon syringe filter of 0.8 μm, 0.4 μm, 0.2 μm, 0.1 μm and 0.05 μm to obtain liposome solution. 10 g maltose, 5 g saccharose and 15 g mannitol were dissolved in the liposome solution by stirring and diluted to 100 mL with water. The pH value was adjusted to 4.21 with phosphoric acid. The liposome was filtrated and sterilized through a 0.22 μm nylon syringe filter. Then, the filtrate was separately packaged and cap-sealed to obtain a liposomal paclitaxel palmitate formulation with a mean particle size of 102.8 nm.

### Example 8

Preparation of Liposomal Paclitaxel Palmitate Formulations

Oil phase was prepared with 0.26 g paclitaxel palmitate, 2.9 g high-purity egg yolk lecithin (EPCS), 0.32 g DSPE-PEG2000 and 3 g propylene glycol. The mixture was dissolved upon heating at 65° C. 65 g water was heated to 65° C. to obtain a water phase. The oil phase was mixed with water phase by stirring to obtain crude liposome. The crude liposome was placed in an extruder, and sequentially passed through a nylon syringe filter of 2.0 μm, 0.6 μm, 0.2 μm, 0.1 μm and 0.05 μm to obtain liposome solution. 10 g maltose, 10 g saccharose and 10 g mannitol were dissolved in the liposome solution by stirring and diluted to 100 mL with water. The pH value was adjusted to 6.50 with sodium hydroxide solution. The liposome was filtrated and sterilized through a 0.22 μm nylon syringe filter. Then, the filtrate was separately packaged and cap-sealed to obtain a liposomal paclitaxel palmitate formulation with a mean particle size of 109.4 nm.

### Example 9

Preparation of Liposomal Paclitaxel Palmitate Formulations

Oil phase was prepared with 0.26 g paclitaxel palmitate, 2.9 g high-purity egg yolk lecithin (EPCS), 0.32 g DSPE-PEG2000, 4 g propylene glycol and 3 g ethanol. The mixture was dissolved upon heating at 40° C. 60 g water was heated to 40° C. to obtain a water phase. The oil phase was mixed with water phase by stirring to obtain crude liposome. The crude liposome was placed in an extruder, and sequentially passed through a nylon syringe filter of 0.4 μm, 0.2 μm, 0.1 μm and 0.05 μm to obtain liposome solution. 10 g maltose and 15 g trehalose were dissolved in the liposome solution by stirring and diluted to 100 mL with water. The pH value was adjusted to 4.69 with hydrochloric acid. The liposome was filtrated and sterilized through a 0.22 μm nylon syringe filter. Then, the filtrate was separately packaged and cap-sealed to obtain a liposomal paclitaxel palmitate formulation with a mean particle size of 110.3 nm.

### Example 10

Preparation of Liposomal Paclitaxel Palmitate Formulations

Oil phase was prepared with 0.26 g paclitaxel palmitate, 2.9 g high-purity egg yolk lecithin (EPCS), 0.32 g DSPE-PEG2000, 1 g propylene glycol and 0.5 g tert-butanol. The mixture was dissolved upon heating at 45° C. 75 g water was to 45° C. to obtain a water phase. The oil phase was mixed with water phase by stirring to obtain a crude liposome. The crude liposome was placed in an extruder, and sequentially passed through a nylon syringe filter of 0.4 μm, 0.2 μm, 0.1 μm and 0.05 μm to obtain liposome solution. 5 g maltose and 10 g trehalose were dissolved in the liposome solution by stirring and diluted to 100 mL with water. The pH value was adjusted to 5.00 with citric acid. The liposome was filtrated was sterilized through a 0.22 μm nylon syringe filter. Then, the filtrate was separately packaged and cap-sealed to obtain a liposomal paclitaxel palmitate formulation with a mean particle size of 106.6 nm.

### Example 11

Preparation of Liposomal Paclitaxel Palmitate Formulations

Oil phase was prepared with 0.4 g paclitaxel palmitate, 4.5 g high-purity egg yolk lecithin (EPCS), 0.5 g DSPE-PEG2000 and 10 g propylene glycol. The mixture was dissolved upon heating at 40° C. 60 g water was heated to 40° C. to obtain a water phase. The oil phase was mixed with water phase by stirring to obtain crude liposome. The crude liposome was placed in an extruder, and sequentially passed through a nylon syringe filter of 1.0 μm, 0.8 μm, 0.4 μm, 0.1 μm and 0.05 μm to obtain liposome solution. The propylene glycol was removed from liposome solution by ultrafiltration. 15 g maltose and 10 g saccharose were dissolved in the liposome solution by stirring and diluted to 100 mL with water. The pH value was adjusted to 4.50 with hydrochloric acid. The liposome was filtrated was sterilized through a 0.22 μm nylon syringe filter. Then the filtrate was separately packaged and cap-sealed to obtain a liposomal paclitaxel palmitate formulation with a mean particle size of 111.8 nm.

### Example 12

Preparation of Liposomal Paclitaxel Palmitate Formulations

Oil phase was prepared with 1 g paclitaxel palmitate, 10 g high-purity egg yolk lecithin (EPCS), 1 g DSPE-PEG2000, 1 g cholesterol and 10 g propylene glycol. The mixture was dissolved upon heating at 75° C. 75 g water was heated to 75° C. to obtain a water phase. The oil phase was mixed with water phase by stirring to obtain a crude liposome. The crude liposome was further emulsified with a high-pressure homogenizer to obtain liposome solution. The propylene glycol was removed from liposome solution by ultrafiltration. The liposome solution was diluted to 100 mL with water. The pH value was adjusted to 8.00 with citric acid. The liposome was filtrated and sterilized through a 0.22 μm nylon syringe filter. Then, the filtrate was separately packaged and cap-sealed to obtain a liposomal paclitaxel palmitate formulation with a mean particle size of 130.0 nm.

### Example 13

Preparation of Liposomal Paclitaxel Palmitate Formulations

Oil phase was prepared with 0.2 g paclitaxel palmitate, 2.3 g high-purity egg yolk lecithin (EPCS), 0.25 g DSPE-PEG2000 and 2 g propylene glycol. The mixture was dissolved upon heating at 70° C. 10 g maltose and 15 g trehalose were dissolved in 70 g of water at 70° C. to obtain water phase. The oil phase was mixed with water phase by stirring to obtain a crude liposome. The crude liposome was placed in an extruder, and sequentially passed through a nylon syringe filter of 0.4 μm, 0.2 μm, 0.1 μm and 0.05 μm to obtain liposome solution. The liposome solution was diluted to 100 mL with water. The pH value was adjusted to 4.80 with hydrochloric acid. The liposome was filtrated was sterilized through a 0.22 μm nylon syringe filter. Then, the filtrate was separately packaged and cap-sealed to obtain a liposomal paclitaxel palmitate formulation with a mean particle size of 104.6 nm.

### Example 14

Preparation of Liposomal Paclitaxel Palmitate Formulations

Oil phase was prepared with 0.2 g paclitaxel palmitate, 2.0 g high-purity egg yolk lecithin (EPCS), 0.1 g DSPE-PEG2000, and 7.0 g propylene glycol. The mixture was dissolved upon heating at 45° C. 10 g maltose and 15 g trehalose were dissolved in 70 g water at 45° C. to obtain water phase. The oil phase was mixed with water phase by stirring to obtain a crude liposome. The crude liposome was placed in an extruder, and sequentially passed through a nylon syringe filter of 0.6 μm, 0.4 μm, 0.2 μm, 0.1 μm and 0.05 μm to obtain liposome solution. The propylene glycol was removed from liposome solution by ultrafiltration. The liposome solution was diluted to 100 mL with water. The pH value was adjusted to 4.00 with citric acid. The liposome was filtrated was sterilized through a 0.22 μm nylon syringe filter. Then, the filtrate was separately packaged and cap-sealed to obtain a liposomal paclitaxel palmitate formulation with a mean particle size of 109.0 nm.

### Example 15

Preparation of Liposomal Paclitaxel Palmitate Formulations

To prepare the liposomal paclitaxel palmitate formulation, an oil phase was first prepared by combining 0.26 g paclitaxel palmitate, 3 g egg phosphatidyl choline (EPCS) and 0.3 g DSPE-PEG2000 with 5 g propylene glycol and 1 g tert-butanol. This mixture was dissolved by stirring and heated at 50° C. to obtain a clear oil phase. The aqueous phase was prepared by dissolving 8 g maltose and 12 g trehalose in 70 g water at 50° C. The oil phase was then added to the aqueous phase by stirring to produce a crude liposome. This crude liposome was passed through an extruder with an extrusion membrane with a pore size of 0.4 μm, 0.2 μm, 0.1 μm, and 0.05 μm to obtain a liposome solution. The liposome was diluted to 100 mL with water and its pH value was adjusted to 4.3 by citric acid. The liposome was sterilized through a 0.22 μm nylon syringe filter. Then, the filtrate was separately packaged, freeze-dried and cap-sealed to obtain a liposomal paclitaxel palmitate formulation lyophilized powder. The mean particle size of the liposome was 95.5 nm.

### Example 16

Preparation of Liposomal Paclitaxel Palmitate Formulations

To prepare the liposomal paclitaxel palmitate formulation, an oil phase was first prepared by combining 0.1 g paclitaxel palmitate, 1 g soyabean lecithin, 0.05 g cholesterol and 0.05 g DSPE-PEG2000 with 1 g propylene glycol. This mixture was dissolved by stirring and heated at 65° C. to obtain a clear oil phase. The aqueous phase was prepared by dissolving 5 g maltose in 85 g water at 65° C. The oil phase was then added to the aqueous phase by stirring to produce a crude liposome. This crude liposome was passed through an extruder with an extrusion membrane with a pore size of 0.2 μm, 0.1 μm, and 0.05 μm to obtain a liposome solution. The liposome was diluted to 100 mL with water and its pH value was adjusted to 8.0 by sodium hydroxide. The liposome was sterilized through a 0.22 μm nylon syringe filter, and then the filtrate was separately packaged, freeze-dried and cap-sealed to obtain a liposomal paclitaxel palmitate formulation lyophilized powder. The mean particle size of the liposome was 86.7 nm.

### Example 17

Preparation of Liposomal Paclitaxel Palmitate Formulations

To prepare the Liposomal paclitaxel palmitate formulation, an oil phase was first prepared by combining 0.7 g paclitaxel palmitate, 3 g hydrogenated soya lecithin (HPSC), 1 g egg yolk phosphatidylcholines, 0.2 g cholesterol and 0.8 g DSPE-PEG2000 with 5 g propylene glycol and 2 g anhydrous ethanol. This mixture was dissolved by stirring and heated at 55° C. to obtain a clear oil phase. The aqueous phase was prepared by heating 80 g water at 55° C. The oil phase was then added to the aqueous phase by stirring to produce a crude liposome. This crude liposome was passed through a high-pressure microfluidizer and passed through an extruder with an extrusion membrane with a pore size of 0.4 μm, 0.1 μm, and 0.05 μm to obtain a liposome solution. Propylene glycol and absolute ethanol were removed from the liposome solution by ultrafiltration. The liposome was then diluted to 100 mL with water and its pH value was adjusted to 7.5 by dipotassium hydrogen phosphate and potassium dihydrogen phosphate. The liposome was sterilized through a 0.22 μm nylon syringe filter, and then the filtrate was separately packaged, charged with nitrogen and cap-sealed to obtain a liposomal paclitaxel palmitate formulation. The mean particle size of the liposome was 117.9 nm.

### Example 18

Preparation of Liposomal Paclitaxel Palmitate Formulations

To prepare the Liposomal paclitaxel palmitate formulation, an oil phase was first prepared by combining 0.7 g Paclitaxel palmitate, 3 g 1,2-dipalmitoyl-sn-glycero-3-phosphocholine (DPPC), 2 g phosphatidylcholine, 5 g phosphatidylethanolamine, 0.5 g cholesterol, and 0.8 g DSPE-PEG2000 with 10 g propylene glycol. This mixture was dissolved by stirring and heated at 55° C. to obtain a clear oil phase. The aqueous phase was prepared by heating 75 g water at 55° C. The oil phase was then added to the aqueous phase by stirring to produce a crude liposome. This crude liposome was passed through a high-pressure microfluidizer and passed through an extruder with an extrusion membrane with a pore size of 0.4 μm, 0.1 μm, and 0.05 μm to obtain a liposome solution. After removing propylene glycol from the liposome solution by ultrafiltration, the liposome was diluted to 100 mL with water and its pH value was adjusted to 7.5 by disodium hydrogen phosphate and sodium dihydrogen phosphate. The liposome was sterilized through a 0.22 μm nylon syringe filter, and then the filtrate was separately packaged, charged with nitrogen, and cap-sealed to obtain a liposomal paclitaxel palmitate formulation. The mean particle size of the liposome was 118.0 nm.

### Example 19

Preparation of Liposomal Paclitaxel Palmitate Formulations

To prepare the Liposomal paclitaxel palmitate formulation, an oil phase was first prepared by combining 0.5 g paclitaxel palmitate, 2 g phosphatidylserines, 1.5 g 1,2-dimyristoyl-sn-glycero-3-phosphocholine (DMPC), 2.5 g 1,2-distearoyl-sn-glycero-3-phosphocholine (DSPC), 0.2 g cholesterol, and 0.5 g DSPE-PEG2000 with 6 g propylene glycol. This mixture was dissolved by stirring and heated at 65° C. to obtain a clear oil phase. The aqueous phase was prepared by heating 70 g water at 65° C. The oil phase was then added to the aqueous phase by stirring to produce a crude liposome. This crude liposome was passed through a high-pressure microfluidizer to obtain a liposome solution. The liposome was diluted to 100 mL with water and its pH value was adjusted to 3.0 by hydrochloric acid. The liposome was sterilized through a 0.22 μm nylon syringe filter, and then the filtrate was separately packaged, charged with nitrogen and cap-sealed to obtain a liposomal paclitaxel palmitate formulation. The mean particle size of the liposome was 115.2 nm.

### Example 20

Preparation of Liposomal Paclitaxel Palmitate Formulations

To prepare the liposomal paclitaxel palmitate formulation, an oil phase was first prepared by combining 0.26 g paclitaxel palmitate, 2.9 g egg phosphatidyl choline (EPCS) and 0.32 g DSPE-PEG2000 with 2 g propylene glycol. This mixture was dissolved by stirring and heated at 70° C. to obtain a clear oil phase. The aqueous phase was prepared by heating 50 g water at 40° C. The oil phase was then added to the aqueous phase by stirring to produce a crude liposome. This crude liposome was passed through an extruder with an extrusion membrane with a pore size of 0.8 μm, 0.4 μm, 0.2 μm, 0.1 μm and 0.05 μm to obtain a liposome solution. The liposome was added with 10 g maltose, 15 g sucrose, and 15 g mannitol, and then liposome was diluted to 100 mL with water and its pH value was adjusted to 6.5 by disodium citrate. The liposome was sterilized through a 0.22 μm nylon syringe filter, and then the filtrate was separately packaged, freeze-dried and cap-sealed to obtain a liposomal paclitaxel palmitate formulation lyophilized powder. The mean particle size of the liposome was 101.7 nm.

### Example 21

Preparation of Liposomal Paclitaxel Palmitate Formulations

To prepare the liposomal paclitaxel palmitate formulation, an oil phase was first prepared by combining 0.2 g paclitaxel palmitate, 3.0 g egg yolk phosphatidylcholines, 2 g sphingomyelin and 0.4 g DSPE-PEG2000 with 3 g propylene glycol and 2 g anhydrous ethanol. This mixture was dissolved by stirring and heated at 25° C. to obtain a clear oil phase. The aqueous phase was prepared by heating 65 g water at 25° C. The oil phase was then added to the aqueous phase by stirring to produce a crude liposome. This crude liposome was passed through an extruder with an extrusion membrane with a pore size of 0.8 μm, 0.4 μm, 0.2 μm, 0.1 μm and 0.05 μm to obtain a liposome solution. The liposome was added with 3 g Lactose, 4 g glucose, 8 g sorbitol and 5 g threonine, and then diluted to 100 mL with water, and its pH value was adjusted to 4.78 by citric acid. The liposome was sterilized through a 0.22 μm nylon syringe filter, and then the filtrate was separately packaged, freeze-dried and cap-sealed to obtain a liposomal paclitaxel palmitate formulation lyophilized powder. The mean particle size of the liposome was 95.8 nm.

### Example 22

Preparation of Liposomal Paclitaxel Palmitate Formulations

To prepare the liposomal paclitaxel palmitate formulation, an oil phase was first prepared by combining 0.2 g paclitaxel palmitate, 3.0 g egg yolk phosphatidylcholines, 2.0 g sphingomyelin, and 0.4 g DSPE-PEG2000 with 4 g propylene glycol and 1 g anhydrous ethanol. This mixture was dissolved by stirring and heated at 25° C. to obtain a clear oil phase. The aqueous phase was prepared by heating 65 g water at 25° C. The oil phase was then added to the aqueous phase by stirring to produce a crude liposome. This crude liposome was passed through an extruder with an extrusion membrane with a pore size of 0.8 μm, 0.4 μm, 0.2 μm, 0.1 μm and 0.05 μm to obtain a liposome solution. The liposome was added with 10 g maltose, 5 g xylitol and 5 g threonine, and then diluted to 100 mL with water, and its pH value was adjusted to 5.50 by citric acid. The liposome was sterilized through a 0.22 μm nylon syringe filter, and then the filtrate was separately packaged, freeze-dried and cap-sealed to obtain a liposomal paclitaxel palmitate formulation lyophilized powder. The mean particle size of the liposome was 70.0 nm.

### Example 23

Preparation of Liposomal Paclitaxel Palmitate Formulations

To prepare the Liposomal paclitaxel palmitate formulation, an oil phase was first prepared by combining 0.4 g paclitaxel palmitate, 4.5 g egg phosphatidyl choline (EPCS) and 0.5 g DSPE-PEG2000 with 6 g propylene glycol. This mixture was dissolved by stirring and heated at 45° C. to obtain a clear oil phase. The aqueous phase was prepared by heating 50 g water at 25° C. The oil phase was then added to the aqueous phase by stirring to produce a crude liposome. This crude liposome was passed through an extruder with an extrusion membrane with a pore size of 0.8 μm, 0.4 μm, 0.2 μm, 0.1 μm and 0.05 μm to obtain a liposome solution. After removing propylene glycol by ultrafiltration, the liposome was added with 10 g maltose, 10 g erythritol and 15 g mannitol, and then diluted to 100 mL with water, and its pH value was adjusted to 3.5 by citric acid. The liposome was sterilized through a 0.22 μm nylon syringe filter, and then the filtrate was separately packaged, freeze-dried and cap-sealed to obtain a liposomal paclitaxel palmitate formulation lyophilized powder. The mean particle size of the liposome was 110.7 nm.

### Example 24

Preparation of Liposomal Paclitaxel Palmitate Formulations

To prepare the Liposomal paclitaxel palmitate formulation, an oil phase was first prepared by combining 0.26 g Paclitaxel palmitate, 2.9 g egg phosphatidyl choline (EPCS) and 0.3 g DSPE-PEG2000 with 5 g propylene glycol. This mixture was dissolved by stirring and heated at 50° C. to obtain a clear oil phase. The aqueous phase was prepared by dissolving 10 g maltose and 15 g trehalose in 65 g water at 30° C. The oil phase was then added to the aqueous phase by stirring to produce a crude liposome. This crude liposome was passed through an extruder with an extrusion membrane with a pore size of 0.8 μm, 0.4 μm, 0.2 μm, 0.1 μm and 0.05 μm to obtain a liposome solution. The liposome was diluted to 100 mL with water, and its pH value was adjusted to 9.0 by sodium hydroxide. The liposome was sterilized through a 0.22 μm nylon syringe filter, and then the filtrate was separately packaged, freeze-dried and cap-sealed to obtain a liposomal paclitaxel palmitate formulation lyophilized powder. The mean particle size of the liposome was 100.5 nm.

Unless otherwise indicated, the numerical ranges involved include the beginning and end values. It will be obvious to those skilled in the art that changes and modifications may be made, and therefore, the aim in the appended claims is to cover all such changes and modifications.

What is claimed is:

1. A liposomal paclitaxel palmitate formulation, comprising:
0.1-1% (w/v) of a paclitaxel palmitate;
1-10% (w/v) of a lecithin;
0.05-1.0% (w/v) of distearoyl phosphoethanolamine-polyethylene glycol 2000 (DSPE-PEG2000); and
water.

2. The formulation of claim 1, being in the form of an aqueous injection.

3. The formulation of claim 1, being in the form of a lyophilized powder.

4. The formulation of claim 1, comprising:
the paclitaxel palmitate: 0.1-1% (g/mL);
the lecithin: 1-10% (g/mL);
cholesterol: 0-1% (g/mL);
the DSPE-PEG2000: 0.05-1.0% (g/mL);
a cryoprotector: 0-40% (g/mL);
a pH modifier adapting to regulate a pH value of the formulation to be 3.0-9.0; and
water.

5. The formulation of claim 1, comprising:
the paclitaxel palmitate: 0.1-0.7% (g/mL);
the lecithin: 1-8% (g/mL);
cholesterol: 0-0.5% (g/mL);
the DSPE-PEG2000: 0.1-0.8% (g/mL);
a cryoprotector: 5-40% (g/mL);
a pH modifier adapting to regulate a pH value of the formulation to be 4.0-8.0; and
water.

6. The formulation of claim 1, comprising:
the paclitaxel palmitate: 0.2-0.5% (g/mL);
the lecithin: 2-6% (g/mL);
cholesterol: 0-0.5% (g/mL);
the DSPE-PEG2000: 0.1-0.5% (g/mL);
a cryoprotector: 10-35% (g/mL);
a pH modifier adapting to regulate a pH value of the formulation to be 4.5-7.5; and
water.

7. The formulation of claim 1, wherein the lecithin is selected from the group consisting of egg phosphatidyl choline (EPCS), hydrogenated soya lecithin, 1,2-dimyristoyl-sn-glycero-3-phosphocholine (DMPC), soyabean lecithin, 1,2-dipalmitoyl-sn-glycero-3-phosphocholine (DPPC), 1,2-distearoyl-sn-glycero-3-phosphocholine (DSPC), phosphatidylcholine, egg yolk phosphatidylcholines, phosphatidylethanolamines, phosphatidylserines, sphingomyelin, or a mixture thereof.

8. The formulation of claim 4, wherein the cryoprotector is selected from the group consisting of maltose, trehalose, sucrose, mannitol, lactose, glucose, sorbitol, xylitol, erythritol, threonine, or a mixture thereof.

9. The formulation of claim 5, wherein the cryoprotector is selected from the group consisting of maltose, trehalose, sucrose, mannitol, lactose, glucose, sorbitol, xylitol, erythritol, threonine, or a mixture thereof.

10. The formulation of claim 6, wherein the cryoprotector is selected from the group consisting of maltose, trehalose, sucrose, mannitol, lactose, glucose, sorbitol, xylitol, erythritol, threonine, or a mixture thereof.

11. The formulation of claim 4, wherein the pH modifier is selected from the group consisting of citric acid, hydrochloric acid, sodium hydroxide, phosphoric acid, disodium hydrogen phosphate, sodium dihydrogen phosphate, dipotassium hydrogen phosphate, potassium dihydrogen phosphate, disodium citrate, tri sodium citrate, or a mixture thereof.

12. The formulation of claim 5, wherein the pH modifier is selected from the group consisting of citric acid, hydrochloric acid, sodium hydroxide, phosphoric acid, disodium hydrogen phosphate, sodium dihydrogen phosphate, dipotassium hydrogen phosphate, potassium dihydrogen phosphate, disodium citrate, tri sodium citrate, or a mixture thereof.

13. The formulation of claim 6, wherein the pH modifier is selected from the group consisting of citric acid, hydrochloric acid, sodium hydroxide, phosphoric acid, disodium hydrogen phosphate, sodium dihydrogen phosphate, dipotassium hydrogen phosphate, potassium dihydrogen phosphate, disodium citrate, tri sodium citrate, or a mixture thereof.

14. The formulation of claim 1, wherein the paclitaxel palmitate, the lecithin, and the DSPE-PEG2000 form a liposome having a particle size of 70-130 nm.

15. A method for preparing a liposomal paclitaxel palmitate formulation comprising 0.1-1% (w/v) of a paclitaxel palmitate, 1-10% (w/v) of a lecithin, 0.05-10% (w/v) of distearoyl phosphoethanolamine-polyethylene glycol 2000 (DSPE-PEG2000), and water, the method comprising:
1) mixing the paclitaxel palmitate, lecithin, cholesterol, DSPE-PEG2000, and an organic solvent, and heating a resulting mixture at 25-75° C. to yield an organic phase;
2) heating an aqueous phase to 25-75° C., and stirring and adding the organic phase to the aqueous phase, to yield a crude liposome;
3) emulsifying the crude liposome, to yield a liposome solution;
4) adding a cryoprotector to the liposome solution, adding water to the liposome solution to reach a preset calibration, adding a pH modifier, filtering the liposome solution, and packaging.

16. The method of claim 15, further comprising preparing the liposomal paclitaxel palmitate formulation into a lyophilized powder.

17. The method of claim 15, wherein the organic solvent is selected from the group consisting of propylene glycol, anhydrous ethanol, tert-butanol, or a mixture thereof.

18. The method of claim 15, wherein a usage amount of the organic solvent is 1-10% (g/mL).

19. The method of claim 15, wherein the liposome solution is filtered using a 0.22 μm nylon syringe filter.

20. The method of claim 15, wherein the crude liposome is emulsified by using an extrusion film having a pore size of 2.0 μm, 1.0 μm, 0.8 μm, 0.6 μm, 0.4 μm, 0.2 μm, 0.1 μm or 0.05 μm.
